(19) United States
(12) Patent Application Publication
(10) Pub. No.: US 2005/0240357 A1
(43) Pub. Date: Oct. 27, 2005

(54) Methods and systems for differential clustering

(76) Inventor: James M. Minor, Los Altos, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/831,866

(22) Filed: Apr. 26, 2004

(51) Int. Cl. G06F 19/00; G01N 33/48; G01N 33/50

(52) U.S. Cl. 702/20

(57) ABSTRACT

Methods, systems and computer readable media for differential clustering of gene expression response profiles for identification of potential functionally variant genes in a high throughput manner. Gene expression data is provided for a number of samples. Gene expression response profiles are generated for various sets of the samples and then differentially clustered across such sets to observe genes whose expression response profiles change cluster membership going from one set to another. Statistical analysis is performed with regard to the change from one cluster membership to another to determine whether the change from one cluster membership to another is statistically significant. If the change is determined to be statistically significant, the gene represented by the gene expression response profiles having been analyzed is identified as being a potential functionally variant gene. The nature of the function change may also be identified by the present systems, methods and computer readable media.

### BACKGROUND OF THE INVENTION

Many genes in an organism impact specific proteins by encoded translations and posterior modifications/variations. Others enable expression regulation by interference tactics, e.g., noise and decoy DNA. Gene expression processes occur in two general steps: transcription and translation as modified by feedback loops. For example, different protein transcription factors bind to promoter sites on the gene to be transcribed. An RNA polymerase binds to the complex of transcription factors, and working together, they open the DNA double helix of the gene. The RNA polymerase then proceeds down one strand of the separated double helix. For protein-encoding genes the nucleosomes in front of the advancing RNA polymerase are removed by a complex of proteins, which also replaces the nucleosomes after the DNA has been transcribed and the RNA polymerase has passed over the strand.

As the RNA polymerase travels along the DNA strand, it assembles ribonucleotides, which are supplied as triphosphates, e.g., ATP) into a strand of RNA. Each ribonucleotide is inserted into the growing RNA strand following the rules of base pairing. Thus for each “C” base encountered on the DNA strand, a “G” base is inserted in the RNA; for each “G” base, a “C” base; and for each “T” base, an “A” base. For each “A” base on the DNA strand, an insertion of a “U” base is made, since there is no “T” base in RNA.

As each nucleoside triphosphate is brought in to add to the end of the growing RNA strand, the two terminal phosphates are removed. When the RNA polymerase encounters a termination signal (a specific sequence of nucleotides), it and its transcript are released from the DNA. A variety of different termination signals are used by the genome.

All the primary transcripts produced in the nucleus must undergo processing steps to produce functional RNA molecules for export to the cytosol. We shall confine ourselves to a view of the steps as they occur in the processing of pre-mRNA to mRNA. FIG. 1 shows a flow path 1 of steps that occur in processing pre-mRNA to mRNA. For a more detailed discussion of transcription, see http://users.rcn.com/ikimball.ma.ultranet/BiologyPages/T/Transcription.html#rn a_processing.

A cap 5 (modified guanine “G” base) is synthesized and attached to the 5′ end of the pre-mRNA as it emerges from processing by the RNA polymerase. The cap protects the RNA from being degraded by enzymes that degrade RNA from the 5′ end. Step-by-step removal of introns 2 present in the pre-mRNA and splicing of the remaining exons 3 is preformed, because generally, genes are split and must be assembled to contain the useful information contained in the exons. Removal of introns takes place as the pre-mRNA continues to emerge from RNA polymerase.

When transcription is complete, the transcript is cut at a site (which may be hundreds of nucleotides before its end), and a stretch of adenine (“A” base) nucleotides, called a poly(A) tail 4 is attached to the exposed 3′ end. This completes the mRNA molecule, which is now ready for export to the cytosol. The remainder of the original transcript is degraded and the RNA polymerase leaves the DNA.

As noted, most genes are split into segments. In decoding the open reading frame of a gene for a known protein, there are generally periodic stretches of DNA calling for amino acids that do not occur in the actual protein product of that gene. Such stretches of DNA, which get transcribed into RNA but not translated into protein, are called introns 2. Those stretches of DNA that do code for amino acids in the protein are called exons 3. Generally, introns tend to be much longer than exons, on average containing orders of magnitude more nucleotides than exons. The cutting and splicing of mRNA must be done with great precision. If even one nucleotide is left over from an intron or one is removed from an exon, the reading frame from that point on will be shifted, producing new codons specifying a totally different sequence of amino acids from that point to the end of the molecule. The removal of introns and splicing of exons is done with what is referred to as a sliceosome, which is a complex of several snRNA molecules and many proteins.

The processing of pre-mRNA for many proteins proceeds along various paths in different cells or under different conditions. For example, early in the differentiation of a B cell (a lymphocyte that synthesizes an antibody) the cell first uses an exon that encodes a transmembrane domain that causes the molecule to be retained at the cell surface. Later, the B cell switches to using a different exon whose domain enables the protein to be secreted from the cell as a circulating antibody molecule.

Alternative splicing provides a mechanism for producing a wide variety of proteins from a small number of genes. Hence whether a particular segment of RNA will be retained as an exon or excised as an intron can vary under different circumstances, and the switching to an alternate splicing pathway must be closely regulated.

Genes that are alternatively spliced to provide different functionalities are generally referred to as functionally variant genes, or simply functional variants. There are many different ways in which genes can become functionally variant. Some examples are splice variants, which were described above, i.e., when the nucleus transcribes the information, it can alter the way it splices the information together; through mutation—e.g., among various races, or by cancer, irradiation etc; and through transcription factors, e.g., the cell machinery sending a message back to the nucleus to instruct the transcription of different proteins. Cancer damage of nuclear chromosomes produces severe changes in functionality of specific genes. Some changes produce metastasis which is usually fatal. In addition to direct transcriptional effects, i.e., single or multi- nucleotide polymorphisms (SNPs or MNPs), feedback transcription factors, etc., most likely there are other causes of functional variants that have not yet even been discovered. For example, there can be variations in the function created by a gene at the post-translation level, where the gene information (RNA) is converted to proteins.

Current methods to detect functionally variant genes are typically inherently slow-throughput, because the approach is generally to somehow first identify the location on the chromosome of mixtures of exons and introns where a modification is thought to occur and then verify the hypothesis through testing. For example, Perlegen Sciences http://www.perlegen.com/ is developing a library of single nucleotide polymorphisms (SNPs) to characterize the single nucleotide polymorphism (SNP) mutations by mapping all occurrences of these in genes (such as those occurring among different races of humans). However, the methods used are very slow and tedious, as first researchers have to identify where the SNPs are occurring on the chromosomes and then verify these regions through experimentation, generally by use of microarrays. Even though microarray technology is used for the verification process, the overall process is still a trial and error, hit or miss, very slow process, particularly with regard to identifying locations of occurrence.

What is needed is a high throughput method to screen candidates that are very likely functionally variant genes. It would further be useful to screen such candidates, not only for SNP's, but for any functionally variant genes. (transcription factor type, splice variants, SNPs, even unknown factors or unknown origins). Whatever the cause, it would still be important to identify genes that change their roles/functionalities, and, where possible, to identify the nature of their functional variance, e.g., change of function versus on/off or activation/deactivation of genes.

### SUMMARY OF THE INVENTION

The present invention provides methods, systems and computer readable media for high throughput identification of potential functionally variant genes. Methods, systems and computer readable media are provided for identifying genes of all types, both direct and indirect, that are potentially functionally variant. Based on gene expression values provided for a plurality of genes for each of a number of tissues wherein the expression values are given for the same genes in each of the number of tissues, methods, systems and computer readable media are provided for differentially clustering gene expression response profiles generated based upon at least a first set of tissues taken from the number of tissues and then from at least a second set of tissues taken from the number of tissues. Comparisons are then made between gene expression response profile members in clusters generated with respect to one of the sets with gene expression response profile members in clusters generated with respect to another of the sets and identification of those members that change cluster membership from a first set to a second set examined are further analyzed. Each identified member is statistically analyzed to determine whether the move of that member from membership in a first cluster to membership in a second cluster is significant relative to the variance within the first and second clusters and the variance between the first and second clusters. If determined to be statistically significant, the gene represented by the member is identified as a potential functionally variant gene. The cluster emphasis is on the synchronization of profile trend variations rather than on shifts in expression levels.

The aforementioned process steps may be carried out with regard to more than two groups, while comparing two groups per iteration, for example. Further, one of the groups may include the entire number of tissue samples, which is referred to as a reference set.

Still further, re-grouping of groups may be performed to gain further perspective on the activity of particular genes.

The present invention further provides methods, systems and computer readable media for identifying the nature of the functional change in an identified potentially functionally variant gene, e.g., whether the change in function was due to transcription factors, a gene going from ambient to expressed or vice versa, SNPs, slice variations, or new or unknown functional changes, for example.

Methods, systems and computer readable media are provided for high throughput identification of genes that are potentially functionally variant, by providing gene expression values for a plurality of genes for each of a number of tissues wherein the expression values are given for the same genes in each of the number of tissues; dividing the number of tissues into at least first and second groups of tissues; generating a gene expression response profile for each gene representative of all gene expression values for that gene across all tissue samples in the first group; clustering the gene expression response profiles generated with respect to the first group; generating a gene expression response profile for each gene representative of all gene expression values for that gene across all tissue samples in the second group; clustering the gene expression response profiles generated with respect to the second group; comparing gene expression response profile members in clusters generated with respect to the first group with gene expression response profile members in clusters generated with respect to the second group and identifying those members that change cluster membership in the second group relative to the first group; statistically calculating whether the move of a member from membership in a first cluster to membership in a second cluster is significant relative to the variance within the first and second clusters and the variance between the first and second clusters; and, if the move is calculated to be significant, identifying the gene represented by the member as a potential functionally variant gene.

The present invention further covers forwarding a result, transmitting data representing a result and/or receiving a result obtained from any of the methods described herein.

These and other advantages and features of the invention will become apparent to those persons skilled in the art upon reading the details of the systems, methods and computer readable media as more fully described below.

### BRIEF DESCRIPTION OF THE DRAWINGS

FIG. 1 shows a flow path 1 of steps that occur in processing pre-mRNA to mRNA.

FIG. 2 is a schematic representation of a matrix of gene expression values generated from n experiments.

FIG. 3 is a schematic representation of a matrix of gene expression values generated from ten experiments, wherein the experiments have been grouped.

FIG. 4A schematically shows an example of clusters aA and aB identified from a clustering process with regard to group 100a in FIG. 3.

FIG. 4B schematically shows re-clustering of the genes from FIG. 3 based upon gene expression response signatures with regard to experiments in group 100b).

FIG. 5 shows a chart reporting clustering results of three reference clusters compared to nine sub-clusters.

FIG. 6 shows a canonical plot of the gene expression signatures from clusters 18 and 7 of FIG. 5.

FIG. 7 is another schematic representation of a process by which gene expression signatures are identified as they jump from one cluster membership in a first group of samples to another cluster membership in a second group of samples.

FIG. 8 is a block diagram illustrating an example of a generic computer system which may be used in implementing the present invention.

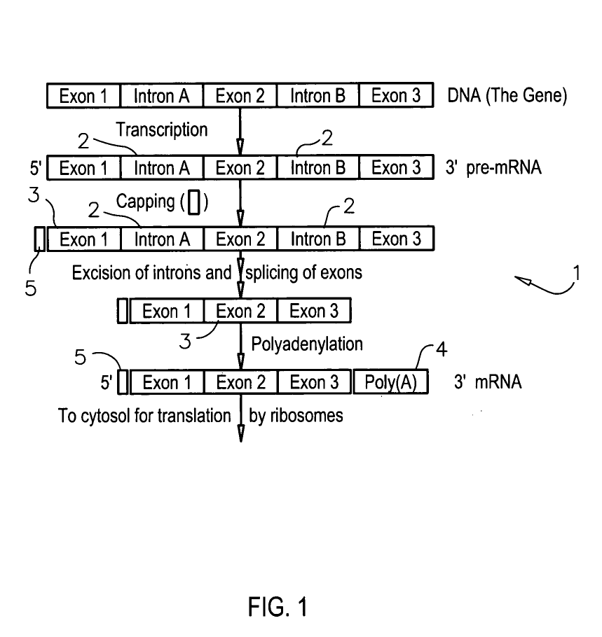

### DETAILED DESCRIPTION OF THE INVENTION

Before the present methods, systems and computer readable media are described, it is to be understood that this invention is not limited to particular examples described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a gene signature” includes a plurality of such gene signatures and reference to “the cluster” includes reference to one or more clusters and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

A “microarray”, “bioarray” or “array”, unless a contrary intention appears, includes any one-, two-or three-dimensional arrangement of addressable regions bearing a particular chemical moiety or moieties associated with that region. A microarray is “addressable” in that it has multiple regions of moieties such that a region at a particular predetermined location on the microarray will detect a particular target or class of targets (although a feature may incidentally detect non-targets of that feature). Array features are typically, but need not be, separated by intervening spaces. In the case of an array, the “target” will be referenced as a moiety in a mobile phase, to be detected by probes, which are bound to the substrate at the various regions. However, either of the “target” or “target probes” may be the one, which is to be evaluated by the other.

Typically a “pulse jet” is a device which can dispense drops in the formation of an array. Pulse jets operate by delivering a pulse of pressure to liquid adjacent an outlet or orifice such that a drop will be dispensed therefrom. Any given substrate may carry one, or more arrays disposed on a front surface of the substrate. A typical array may contain more than ten, more than one hundred, more than one thousand, more than ten thousand features, or even more that one hundred thousand features, in an area of less that 20 cm² or even less that 10 cm². For example, features may have widths in the range from about 10 μm to 1.0 cm. In other embodiments, each feature may have a width (that is, diameter for a round spot) in the range of about 1.0 μm to 1.0 mm, and more usually about 10 μm to 200 μm. Non-round features may have area ranges equivalent to that of circular features with the foregoing with ranges. At least some, or all, of the features are of different compositions, each feature typically being of a homogeneous composition within the feature. Interfeature areas will typically be present which do not carry chemical moiety of a type of which the features are composed. Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations. Methods to fabricate arrays are described in detail in U.S. Pat. Nos. 6,242,266; 6,232,072; 6,180,351; 6,171,797 and 6,323,043. As already mentioned, these references are incorporated herein by reference. Other drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

Following receipt by a user, an array will typically be exposed to a sample and then read. Reading of an array may be accomplished by illuminating the array and reading the location and intensity of resulting fluorescence at multiple regions on each feature of the array. For example, a scanner may be used for this purpose is the AGILENT MICROARRAY SCANNER manufactured by Agilent Technologies, Palo, Alto, Calif. or other similar scanner. Other suitable apparatus and methods are described in U.S. Pat. Nos. 6,518,556; 6,486,457; 6,406,849; 6,371,370; 6,355,921; 6,320,196; 6,251,685 and 6,222,664. However, arrays may be read by any other methods or apparatus than the foregoing, other reading method including other optical techniques or electrical techniques (where each feature is provided with an electrode to detect bonding at that feature in a manner disclosed in U.S. Pat. Nos. 6,251,685, 6,221,583 and elsewhere).

A “gene expression signature” or “gene expression profile”, refers to a gene expression profile over a number of genes, typically from the same sample, which may include all of the genes being measured for that sample, or a selected number of those genes. Specific gene expression signatures can often identify specific events occurring within a cell.

A “gene expression response signature” or “gene expression response profile” refers to a profile generated by expression values of the same gene over a number of samples.

When one item is indicated as being “remote” from another, this is referenced that the two items are at least in different buildings, and may be at least one mile, ten miles, or at least one hundred miles apart.

“Communicating” information references transmitting the data representing that information as electrical signals over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

A “processor” references any hardware and/or software combination which will perform the functions required of it. For example, any processor herein may be a programmable digital microprocessor such as available in the form of a mainframe, server, or personal computer. Where the processor is programmable, suitable programming can be communicated from a remote location to the processor, or previously saved in a computer program product. For example, a magnetic or optical disk may carry the programming, and can be read by a suitable disk reader communicating with each processor at its corresponding station.

Reference to a singular item, includes the possibility that there are plural of the same items present.

“May” means optionally.

Methods recited herein may be carried out in any order of the recited events which is logically possible, as well as the recited order of events.

All patents and other references cited in this application, are incorporated into this application by reference except insofar as they may conflict with those of the present application (in which case the present application prevails).

The present invention provides methods, systems and computer readable media for high throughput techniques of finding functionally variant genes. Although the examples described refer to the use of microarrays for the high throughput techniques, the present invention is not limited or restricted to the use of microarrays, as any other tools that may be used to input biological samples to which will output gene expression levels may be used, including beads or other tools.

The methods, systems and computer readable media disclosed herein are further important for array probe design, since variant genes may distort the usual clustering processes used in array probe design. Array design focuses on specificity of the ensemble of probes contained on the array. Hence, functional variants cloud the primary objective of specificity. Thus, by identifying variant genes in accordance with the principles described herein, array probe designers may take the identified genes into consideration to enable proper corrections and/or design alternatives of probes.

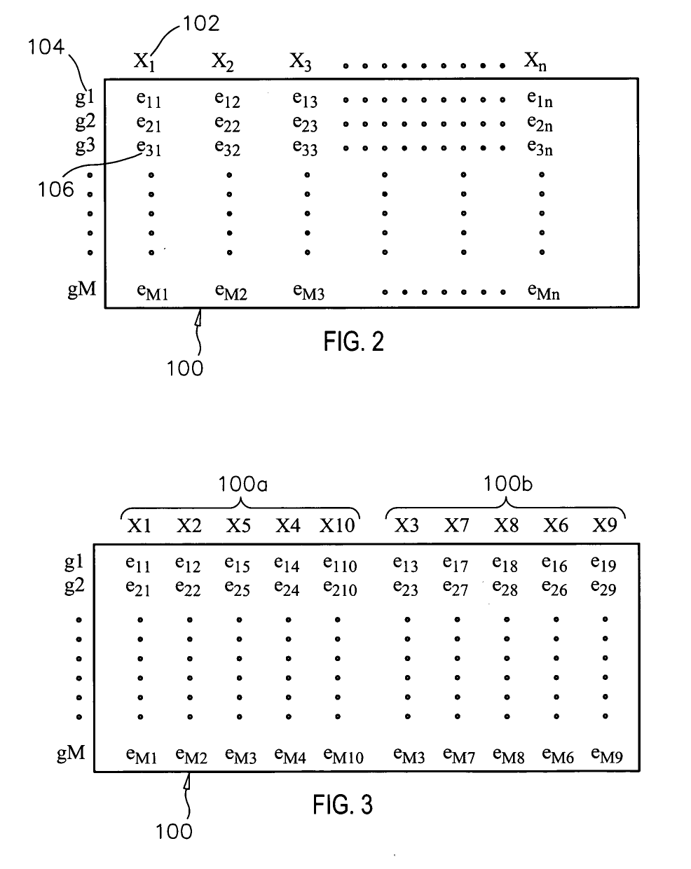

Referring now to FIG. 2, a matrix 100 of gene expression values generated from n experiments is shown. Each experiment may be based on a different tissue sample for example. The number n is a positive integer which may vary. For each experiment 102 (i.e., X1, X2, X3, . . . , Xn) a microarray was run on M genes 104 (i.e., g1, g2, g3, . . . , gM), where M is a positive integer which is generally very large, and may be on the order of 30,000 to 50,000, for example. However, the present invention is not limited to this range, as more or fewer genes could be experimented on according to the principles described herein. For each gene 104 in each experiment 102, an expression value 106e (e.g., e11, el2, e,21, etc.) (or differential expression value) is generated and values 106 make up matrix 100 as shown. Each row of matrix 100 may be used to generate a gene expression response signature or profile across all samples/experiments.

For each experiment 102 run, a gene expression signature or gene expression profile can be generated by plotting the expression values 106 for a single experiment 102 against the genes 104 from which the levels where generated. Thus a gene expression signature is generated from a column of values from matrix 100. Alternatively, for each gene 104, a gene expression response signature or gene expression response profile can be generated by plotting the expression values 106 for a single gene 104 against the experiments 102 from which the levels were generated. Thus a gene expression response signature is generated from a row of values from matrix 100.

The present invention makes use of gene expression response signatures to compare amongst gene expression response signatures and perform various clustering manipulations to identify genes that appear to be changing functionality under varying conditions. The experiments 102 used to generate matrix 100 may be “natural samples” from which that natural variation (inherent variability) in the samples may be taken advantage of to study variations in functionalities of genes, or the experiments may be specifically designed under specific conditions to leverage the kinds of biological information desired to be observed by the experiments. The latter approach generally requires consultation with one or more biological experts to design the biological samples and groups (using statistical design of experiments) to leverage functional variation in genes.

One technique for applying the present principles, whether the experiments are natural samples or specifically designed experiments, involves separating matrix 100 into groups 100a, 100b, . . . , 100n of experiments, as shown in FIG. 3, which would be expected to exhibit variations in functionalities of genes among the various groups. When designing the experiments, these groups can also be predesignated based on best available current knowledge/expertise. With natural samples, the experimenter may be able to subdivide the matrix according to known phenotypic properties of the experiment (e.g., when one group of samples is known to have one type of cancer, another group of samples is known to have another type of cancer, and a third group is known to be non-cancerous), or groups may be identified by comparing gene expression signatures among the experiments and clustering or grouping those experiments found to have gene expression signatures similar to one another.

For simplicity, FIG. 3 shows only two groups 100a and 100b, each with five experiments. Of course, the invention is in no way limited to use of only two groups, the groups do not need to have equal numbers of experiments, and the groups do not need to be limited to five or any set number of experiments. However, the greater number of experiments that there are in the group, the more information this provides for constructing gene expression response signatures and the more sensitivity the process has for comparing these signatures. In general it is useful to have at least five experiments for forming each gene expression response signature, more preferably at least ten experiments, in order to provide good sensitivity when comparing gene response signatures. The bigger that the profiles are, the less chance there is for false positive results.

The gene expression response signatures are next used to perform differential clustering evaluation to identify potentially functionally variant genes in a very fast, high-throughput manner. In this example, the genes in each group (e.g., 100a, 100b) are first clustered with respect to each group. That is, the genes in group 100a are clustered with regard to gene expression response signatures generated from experiments X1,X2,X5,X4,X10, and the genes are again clustered with regard to group 100b using gene expression response signatures generated from experiments X3,X7,X8,X6,X9. The clustering may be performed by conventional clustering techniques, such as by techniques based upon similarity between gene expression response signatures, including similarity metrics based on Pearson's Correlation Coefficient, or other known similarity metrics. Thus, a cluster of profiles is identified based upon the characteristics of profile fuzziness, relative to distance from the center of the nearest cluster. Such processing may be carried out using DynaCluster™, which is discussed in detail in co-pending, commonly owned application Ser. No. 09/986,746, filed Nov. 9, 2001 and titled “System and Method for Dynamic Data Clustering”, or using other readily available clustering applications. application Ser. No. 09/986,746 is hereby incorporated herein, in its entirety, by reference thereto.

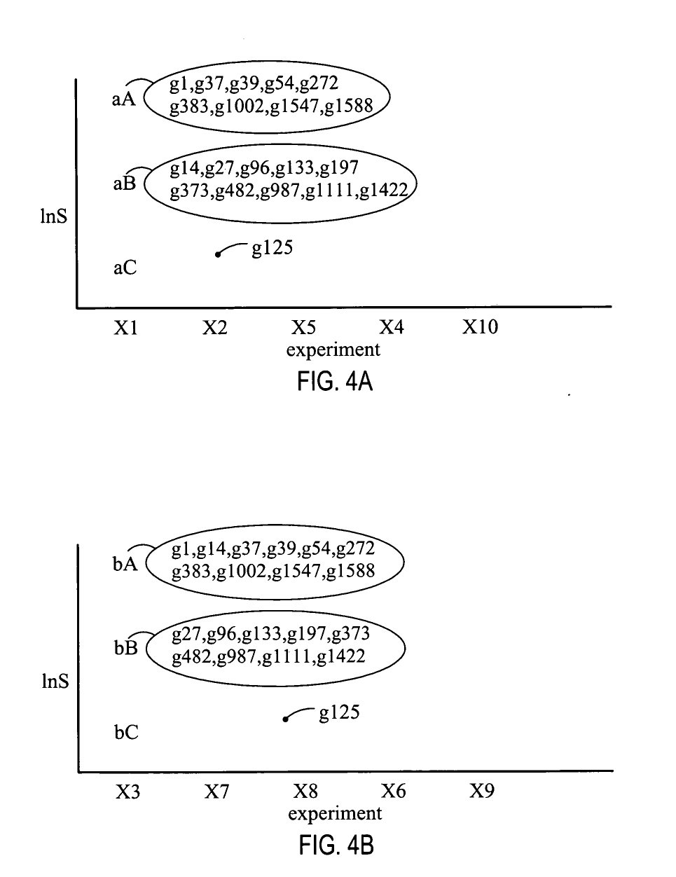

FIG. 4A is a symbolic representation showing an example of clusters aA and aB identified from the clustering process with regard to group 100a. Note that this shows cluster that have significant membership and is for exemplary purposes only, as many more (or fewer) clusters might be identified during the clustering procedure described. If ab initio, the procedure gives one or more clusters each containing only one (or two, or other low number) gene expression response signature, such as cluster aC shown in FIG. 4A, this may indicate that this particular gene is “not comfortable’ (i.e., not similar) to any of the clustered groups of genes. One reason for such a result could be that the single-clustered gene in the example of FIG. 4A is performing like those in cluster aA for experiments X1,X2 and X5, while performing more like genes in cluster aB in experiments X4 and X5, due to a variation in the function of the single gene between experiments. However, this is not the only explanation, as the singularity cluster (single gene) may just be performing dissimilarly to both clusters aA and aB throughout all of the experiments, for example. However, at least ab initio, the system considers such low member clusters to be potential candidates for functionally variant genes, since it does not want to rule out the possibility of the first explanation provided above.

FIG. 4B symbolically shows the re-clustering of the genes based upon gene expression response signatures with regard to experiments X3,X7,X8,X6,X9 (i.e., group 100b). In these results it can be noted that the clusters bA, bB and bC are fairly consistent when comparing them with cluster aA, aB and aC, except that gene g14 is now grouped in the A cluster (i.e., bA), whereas in the previous clustering procedure, gene g14 showed up in the B cluster (aB). This result may indicate that gene g14 has changed function with regard to the experiments in group 100b as compared to its function with regard to the experiments in group 100a. The present techniques do not identify the functions that the gene may be switching between, but only identifies potentially functionally variant genes, based on the jump from being associated with one cluster to being associated with another cluster. Once identified, data representative of the genes identified as being potentially functionally variant may be stored by the system and or used in other processes to further investigate/verify whether those genes identified are in fact functional variants.

After identifying candidate genes that may be functionally variant, further techniques are applied to determine whether the jump that each candidate has made is significant to make a determination that that particular gene has changed functions. Typically statistical techniques are applied at this time which will conclude whether a jump has been significant. In this example, an F-test was used to calculate the variance between clusters divided by the variance within clusters and this value was used as a threshold to determine whether the change of membership of a particular gene from one of those clusters to another was significant, i.e., exceeded the calculated value, to conclude that the gene is potentially changing functions (i.e., a functional variant) by virtue of its changing from one cluster to another.

Thus, the F-test was used to calculate values between each pair of clusters being compared (i.e., clusters A (aA and bA) and B (aB and bB). The scatter or “fuzziness” of each cluster is characterized by the calculated variance of each. If the distance of the jump made is beyond the scatter of the clusters, as determined by well-established statistical methods for determining such, such as the F-test described previously (or using JMP*SAS, available from JMP Software, Cary, N.C.), then the jump is determined to be significant and the conclusion is made that the gene being studied has changed functionality. As noted above, ab initio singularities are considered to inherently be suspected functional variants. Since the scatter about clusters being compared will generally not be the same (e.g., scatter about aA is not the same as scatter about bA), established statistical tests are applied that account for the unequal scatter (i.e., “fuzziness”) when making such comparisons. If the scatter from the clusters A and B (i.e., aA and aB or bA and bB) overlap, then the system typically combines clusters A and B into a single cluster and uses the combined cluster for comparison with other clusters where scatters do not overlap.

Most genes will typically be found not to jump between these designed clusters. This is beneficial to the process, as the comparison of the genes that do jump is based upon the cluster characteristics formed by the genes that stay together (i.e., stay in the same cluster). However, it is important to identify those genes that do jump or switch as they may be changing functionalities in response to a disease or a drug or other stimulus.

A p-value may be assigned to each gene that has made a jump. The p-value may be based on the distance of the jump relative to the scatter distances described above, which is essentially a comparison of between group noise and within group noise, which comparison is performed based on well-known statistical methods. A determination is made as to whether the between group noise is significantly greater than the within group noise. If it is, the conclusion is that the gene has changed functionalities.

Significance depends upon the size of the jump as compared to the distance between the clouds formed by the clusters. A standard t-test can be used to determine significance:
T(df)=Delta/(Delta-standard error)
where
df=degrees of freedom, determined by the number of data points used in determining standard error, and
Delta=size of jump.

Typical statistical normalization is used to generalize the process.

Another approach to performing differential clustering evaluation to identify potentially functionally variant genes in a very fast, high-throughput manner involves clustering the entire dataset, e.g. clustering with respect to all ten experiments in matrix 100 of FIG. 3, to form reference or grand clusters based on clustering operations performed on gene expression response signatures formed from the data in all the experiments. Additionally, clustering is performed on groups of experiments, such as in the manner as described above with regard to FIGS. 3-4B. Then comparisons between the group clusters are made with the reference clusters (e.g., aA and aB compared with referenceA and referenceB, then bA and bB with referenceA and referenceB, and so forth, if there are other groups to compare) to determine whether any genes have jumped from one cluster to another, and the same type of processing is performed to determine whether a jump is significant to conclude that a gene has changed functionality. The reference clusters provide further assurance for consistency in genes that do not switch between groups which may provide a more stable reference against which the jumps are measured.

Still further, the experiments in matrix 100 may be broken down into different groups than those already processed. Thus, in the example referred to in FIG. 3. The groups 100a and 100b may be further divided to form additional groups (ignoring the fact that the example does not show enough experiments to provide additional groups having sufficient members to provide gene expression response signatures from at least five experiments each), with clustering performed on each of the groups, to get a different perspective, which may identify different or additional candidates. Or the same number of groups may be used, but while dividing the groups to have different experiment members. Results of all these variations on processing may then be compared and intersected to provide a “best list” of candidates for functionally variant genes.

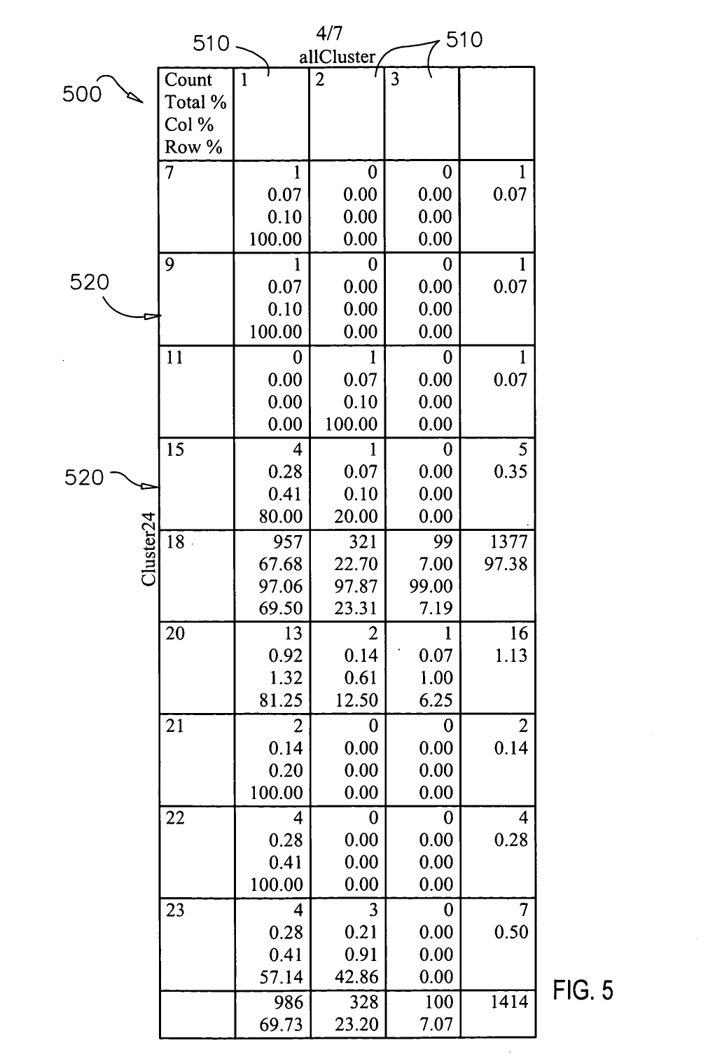

FIG. 5 shows a chart 500 reporting clustering results of three reference clusters 510 that resulted from clustering gene expression response signatures generated from sixty cell lines of a variety of cancer tissues, versus nine clusters 520. It can be observed that the majority of expression signatures (i.e., count=957) in cluster 18 of the melanoma clusters linked with reference cluster 1. However, four expression signatures jumped to cluster 15 two expression signatures jumped to cluster 21, one expression signature jumped to cluster 9, one expression signature jumped to cluster 7, thirteen expression signatures jumped to cluster 20, two expression signatures jumped to cluster 21, four expression signatures jumped to cluster 22 and four expression signatures jumped to cluster 23. In this example, all clustering was performed using DynaClusterm to identify viable clusters, which are then refined by conventional methods, such as K-means clustering. Profile errors were smoothed with a smoothing algorithm described in detail in co-pending, commonly assigned application Ser. No. 10/400,372 filed march 27, 2003 and titled “Method and System for Predicting Multi-Variable Outcomes” and in Application Ser. No. 60/368,586 filed Mar. 29, 2002. Both application Ser. No. 10/400,372 and Application Ser. No. 60/368,586 are hereby incorporated herein, in their entireties, by reference thereto.

Thus, reference cluster 1 appears to be the most common reference cluster to the melanoma clusters overall, and cluster 18 tends to align with reference cluster 1. Therefore, the data was next examined to identify the jumps from cluster 18, relative to reference cluster 1, and were identified as listed above and as shown in column 1 of table 500. For each signature that jumped from cluster 18 to another cluster, the degree of jump was evaluated with respect to the fuzziness of cluster 18 and the fuzziness of the cluster that the particular gene expression response signature jumped to, unless the gene expression response signature that jumped established itself as a singularity. If a gene expression signature jumps and establishes itself as a singularity, then there can be no fuzziness, since it is a “cluster” of one response signature. As noted above, a singularity is treated as a suspected functional variant.

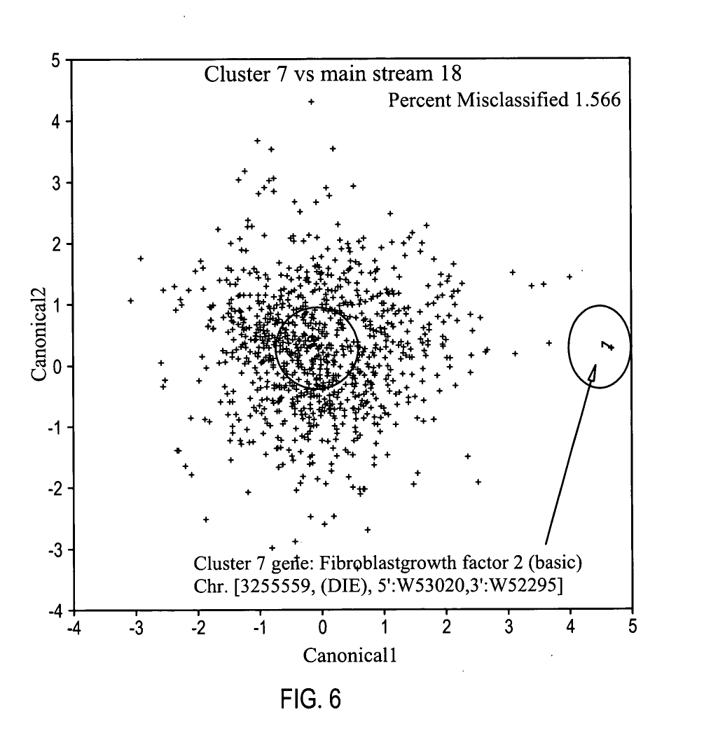

Using a discriminant analysis program, such as JMP*SAS, for example, an estimate of false positive is obtained. That is, for a p-value of 0.01566 found using a discriminant analysis program, this estimates that 1.566% of all signatures may be misclassified by the clustering process. FIG. 6 shows a plot of the gene expression signatures from clusters 18 and 7 of table 500 using Canonical Analysis, wherein JMP*SAS determined p=0.01566 as the estimated false-positive rate. The 50% contour line surrounding the gene expression response signature for cluster 7 implies the degree of confidence for p=0.01566. The discriminant-analysis-generated 50% contours capture 50% of member population for the clusters, based on the centroids of the respective clusters that they surround. Thus, in this example, it was determined that the expression response signature that jumped from cluster 18 to cluster 7 was the result of a functionally variant gene changing functions. Generally, the threshold for significance used is p<.05.

It should be noted that the present invention, while described mainly with regard to comparing clusters from two groups of samples or experiments; or comparing clusters formed from a subset of samples to reference clusters formed from the entire class of samples; is not limited to these examples. While two series of experiments may be compared one to the other, as noted, multiple subgroups can be processed, each to form sets of clusters, and these subset can be compared to reference clusters. Alternatively, or additionally, any subset of groups of experiments may be selected to develop clusters that are common to the selected subset and them compared with reference clusters to see how the cluster/group memberships change during the comparison. By identifying those gene expression response signatures that change or jump, further processing can be done with respect to the identified signatures to determine whether their jumps were significant to conclude that these signatures identify potentially functionally variant genes under the conditions that were present for the experiments.

The present invention is not only useful in identifying potentially functionally variant genes, but may also be useful, in certain instances to determine the type of potentially functionally variant gene that has been identified. For example, a gene may be dormant in one group (e.g., cluster formed from one set of experiments) and them become active in another group and, as such, be identified by the present techniques as a potentially functionally variant gene. By further analysis of the clusters from the first and second groups that the gene expression response signatures from this gene were members in, the analysis would determine that the cluster from the first group has very little variation in expression levels, since this is a cluster of dormant genes. On the other hand, the cluster from the second group will have more variation, since the gene is active in that instance. Therefore, the analyst could determine that the functional variation in this example is from dormancy to playing some form of active role. Identifications of other types of functional variations may also be possible by skilled researchers through the analysis of the properties of the members of the clusters being examined, e.g., the types of genes in a cluster, the expression levels in the expression response signatures representative of the genes in the cluster, etc.

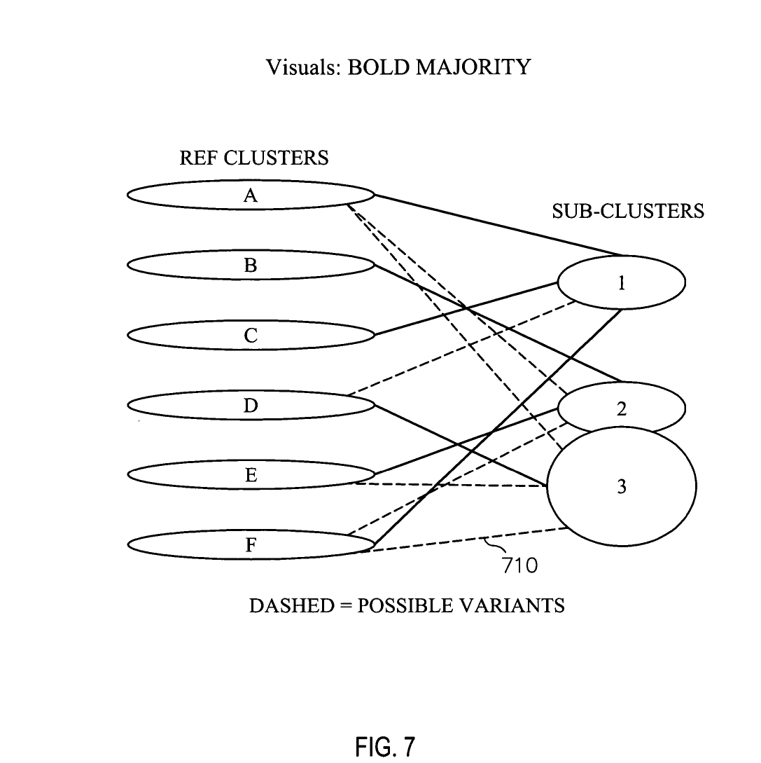

FIG. 7 is another schematic representation of a process by which gene expression signatures are identified as they jump from one cluster membership in a first group of samples to another cluster membership in a second group of samples. In this example, the first group of samples is made up of all the samples being examined, so as to form reference clusters A,B,C,D,E and F. A subset of the first group of samples was then selected to form sub-clusters 1, 2 and 3. Reference clusters A-F were generated as robust clusters by a re-clustering after initial cluster to down weight relatively small clusters. As noted before, extremely small clusters, such as singlets, or clusters having only two or three gene expression response signature members may be considered to be representative of potentially functionally variant genes ab initio.

Reference clusters tend to combine when clustering sub-profiles, due to the lower dimensionalities of the gene expression response signatures from the subset, relative to the total set of samples/experiments. This is evident when comparing the number of sub-clusters (i.e., three) in FIG. 7 to the number of reference clusters (i.e., six). Gene expression response signatures from variant genes tend to deviate from the majority in reference clusters. Such deviations are indicated by the dotted arrows in FIG. 7. The gene expression response signatures represented by the dotted arrows are then processed, as described above, with reference to the clusters involved, to identify those that have jumped significantly, and can therefore be identified as representing potentially functionally variant genes under these conditions. For example, processing of the gene expression response signature represented by dotted arrow 710 would be carried out with reference to the properties of reference cluster F and sub-cluster 3.

The present invention, in addition to the benefits provided in identifying functionally variant genes, is also useful for improving standard clustering operations. By identifying genes which do switch clusters, these can be taken into account and possibly filtered out from standard clustering operations to provide more accurate results for clustering the remainder of the genes that are not functionally variant.

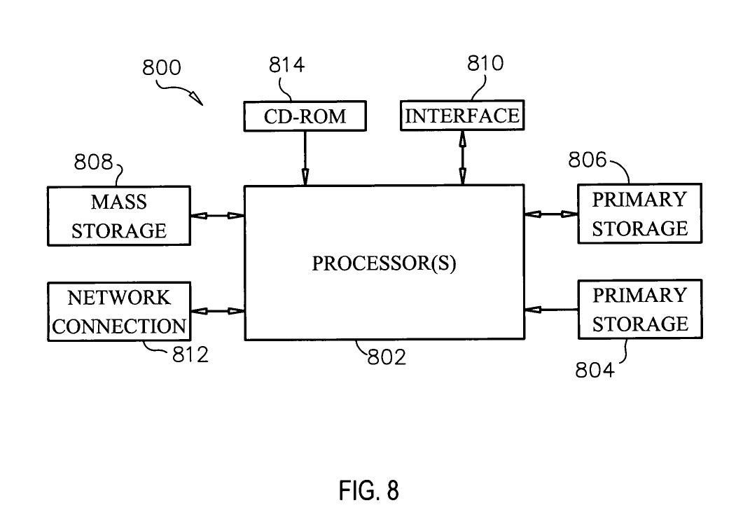

FIG. 8 illustrates a typical computer system in accordance with an embodiment of the present invention. The computer system 800 includes any number of processors 802 (also referred to as central processing units, or CPUs) that are coupled to storage devices including primary storage 806 (typically a random access memory, or RAM), primary storage 804 (typically a read only memory, or ROM). As is well known in the art, primary storage 804 acts to transfer data and instructions uni-directionally to the CPU and primary storage 806 is used typically to transfer data and instructions in a bi-directional manner Both of these primary storage devices may include any suitable computer-readable media such as those described above. A mass storage device 808 is also coupled bi-directionally to CPU 802 and provides additional data storage capacity and may include any of the computer-readable media described above. Mass storage device 808 may be used to store programs, data and the like and is typically a secondary storage medium such as a hard disk that is slower than primary storage. It will be appreciated that the information retained within the mass storage device 808, may, in appropriate cases, be incorporated in standard fashion as part of primary storage 806 as virtual memory. A specific mass storage device such as a CD-ROM 814 may also pass data uni-directionally to the CPU.

CPU 802 is also coupled to an interface 810 that includes one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 802 optionally may be coupled to a computer or telecommunications network using a network connection as shown generally at 812. With such a network connection, it is contemplated that the CPU might receive information from the network, or might output information to the network in the course of performing the above-described method steps. The above-described devices and materials will be familiar to those of skill in the computer hardware and software arts.

The hardware elements described above may implement the instructions of multiple software modules for performing the operations of this invention. For example, instructions for population of stencils may be stored on mass storage device 808 or 814 and executed on CPU 808 in conjunction with primary memory 806.

In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM, CDRW, DVD-ROM, or DVD-RW disks; magneto-optical media such as floptical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular model, tool, process, process step or steps, to the objective, spirit and scope of the present invention. All such modifications are intended to be within the scope of the claims appended hereto.

1. A high throughput method of identifying genes that are potentially functionally variant, said method comprising the steps of:
providing gene expression values for a plurality of genes for each of a number of tissues wherein the expression values are given for the same genes in each of the number of tissues;
dividing the number of tissues into at least first and second groups of tissues;
generating a gene expression response profile for each gene representative of all gene expression values for that gene across all tissue samples in the first group;
clustering the gene expression response profiles generated with respect to the first group;
generating a gene expression response profile for each gene representative of all gene expression values for that gene across all tissue samples in the second group;
clustering the gene expression response profiles generated with respect to the second group;
comparing gene expression response profile members in clusters generated with respect to the first group with gene expression response profile members in clusters generated with respect to the second group and identifying those members that change cluster membership in the second group relative to the first group;
statistically calculating whether the move of a member from membership in a first cluster to membership in a second cluster is significant relative to the variance within the first and second clusters and the variance between the first and second clusters; and
if the move is calculated to be significant, identifying the gene represented by the member as a potential functionally variant gene.

2. The method of claim 1, further comprising identifying small clusters having less than a predefined number of gene expression response signature members resultant from said clustering steps and identifying any genes represented by members in the small clusters as potential functionally variant genes.

3. The method of claim 1, further comprising identifying single gene expression response profiles that do not cluster with clusters produced by said clustering steps and identifying each gene represented by each identified single gene expression response profile as a potential functionally variant gene.

4. The method of claim 1, wherein said dividing comprises dividing the number of tissues into more than two groups of tissues, and wherein said clustering and said generating a gene expression response profile steps are performed with respect to each group, the results of which are compared with each of said first and second groups and with each other to identify those members that change cluster membership when comparing any one group to another, and wherein said statistical calculation is performed with respect to those groups considered, when a member changes cluster membership, to determine if the move was statistically significant.

5. The method of claim 1 wherein each group includes at least five tissue samples.

6. The method of claim 1, further comprising the steps of:
generating a gene expression response profile for each gene representative of all gene expression values for that gene across all tissue samples in the number of tissues;
clustering the gene expression response profiles generated with respect to the totality of the number of tissues;
comparing gene expression response profile members in clusters generated with respect to the totality of the number of tissues with gene expression response profile members in clusters generated with respect to each of said at least first and second groups, respectively, and identifying those members that change cluster membership when comparing clusters generated with respect to the totality of the number of tissues with clusters generated from one of said groups;
statistically calculating whether the move of a member from membership in a first cluster to membership in a second cluster is significant relative to the variance within the first and second clusters and the variance between the first and second clusters; and
if the move is calculated to be significant, identifying the gene represented by the member as a potential functionally variant gene.

7. The method of claim 1, further comprising:
regrouping the number of tissues into different groups of tissues comprising at least different first and different second groups of tissues which are different from said at least first and second groups of tissues; and
carrying out said clustering, generating comparing, statistically calculating and identifying steps with regard to said at least different first and different second groups of tissues to identify potential functionally variant genes.

8. The method of claim 7, further comprising comparing identifications made in claim 1 with identifications made in claim 7.

9. The method of claim 8, further comprising intersecting identifications made in claim 1 with identifications made in claim 7 to form an optimized list of potential functionally variant genes.

10. A method comprising forwarding a result obtained from the method of claim 1 to a remote location.

11. A method comprising transmitting data representing a result obtained from the method of claim 1 to a remote location.

12. The method of claim 1, further comprising determining a specific function that is varying in the identified potentially functionally variant gene, based on at least one of:
characteristics of the first and second clusters in which the member representative of the potentially functionally variant gene had membership, and characteristics of the members in the first and second clusters in which the member representative of the potentially functionally variant gene had membership.

13. A method comprising receiving a result obtained from a method of claim 1 from a remote location.

14. A high throughput method of identifying genes that are potentially functionally variant, said method comprising the steps of: providing gene expression values for a plurality of genes for each of a number of tissues wherein the expression values are given for the same genes in each of the number of tissues;
differentially clustering gene expression response profiles generated based upon at least a first set of tissues taken from the number of tissues and then from at least a second set of tissues taken from the number of tissues;
comparing gene expression response profile members in clusters generated with respect to one of said sets with gene expression response profile members in clusters generated with respect to another of said sets and identifying those members that change cluster membership in said another of said sets relative to said one of said sets;
statistically calculating whether the move of a member from membership in a first cluster to membership in a second cluster is significant relative to the variance within the first and second clusters and the variance between the first and second clusters; and
if the move is calculated to be significant, identifying the gene represented by the member as a potential functionally variant gene.

15. The method of claim 14, wherein said first set includes the total number of tissues and said second set is a subset of the total number of tissues.

16. The method of claim 14, wherein said first set comprises a subset of the total number of tissues and said second set comprises a different subset of the total number of tissues.

17. The method of claim 14, wherein one of sets includes the total number of tissues, and a plurality of sets further include different subsets of the total number of tissues.

18. The method of claim 14, further comprising determining a specific function that is varying in the identified potentially functionally variant gene, based on at least one of: characteristics of the first and second clusters in which the member representative of the potentially functionally variant gene had membership, and characteristics of the members in the first and second clusters in which the member representative of the potentially functionally variant gene had membership.

19. A method comprising forwarding a result obtained from the method of claim 14 to a remote location.

20. A method comprising transmitting data representing a result obtained from the method of claim 14 to a remote location.

21. A method comprising receiving a result obtained from a method of claim 14 from a remote location.

22. A system for high throughput identification of genes that are potentially functionally variant, said system comprising:
means for differentially clustering gene expression response profiles generated from expression values taken from at least a first set of tissues taken from a dataset providing gene expression values for a number of tissues and then clustering gene expression response profiles generated from expression values taken from at least a second set of tissues taken from the number of tissues;
means for comparing gene expression response profile members in clusters generated with respect to one of said sets with gene expression response profile members in clusters generated with respect to another of said sets and identifying those members that change cluster membership in said another of said sets relative to said one of said sets;
means for statistically calculating whether the move of a member from membership in a first cluster to membership in a second cluster is significant relative to the variance within the first and second clusters and the variance between the first and second clusters; and
means for identifying a gene as a potential functionally variant gene if the move is calculated to be significant.

23. A computer readable medium carrying one or more sequences of instructions for high throughput identification of genes that are potentially functionally variant, wherein execution of one or more sequences of instructions by one or more processors causes the one or more processors to perform the steps of:
dividing gene expression values, provided for a plurality of genes for each of a number of tissues wherein the expression values are given for the same genes in each of the number of tissues, into at least first and second groups of tissues;
generating a gene expression response profile for each gene representative of all gene expression values for that gene across all tissue samples in the first group;
clustering the gene expression response profiles generated with respect to the first group;
generating a gene expression response profile for each gene representative of all gene expression values for that gene across all tissue samples in the second group;
clustering the gene expression response profiles generated with respect to the second group;
comparing gene expression response profile members in clusters generated with respect to the first group with gene expression response profile members in clusters generated with respect to the second group and identifying those members that change cluster membership in the second group relative to the first group;
statistically calculating whether the move of a member from membership in a first cluster to membership in a second cluster is significant relative to the variance within the first and second clusters and the variance between the first and second clusters; and
if the move is calculated to be significant, identifying the gene represented by the member as a potential functionally variant gene.

24. A computer readable medium carrying one or more sequences of instructions for high throughput identification of genes that are potentially functionally variant, wherein execution of one or more sequences of instructions by one or more processors causes the one or more processors to perform the steps of:
differentially clustering gene expression response profiles, provided for a plurality of genes for each of a number of tissues wherein the expression values are given for the same genes in each of the number of tissues, generated based upon at least a first set of tissues taken from the number of tissues and then from at least a second set of tissues taken from the number of tissues;
comparing gene expression response profile members in clusters generated with respect to one of said sets with gene expression response profile members in clusters generated with respect to another of said sets and identifying those members that change cluster membership in said another of said sets relative to said one of said sets;
statistically calculating whether the move of a member from membership in a first cluster to membership in a second cluster is significant relative to the variance within the first and second clusters and the variance between the first and second clusters; and
if the move is calculated to be significant, identifying the gene represented by the member as a potential functionally variant gene.
